(19) United States
(12) Patent Application Publication
(10) Pub. No.: US 2019/0125712 A1
(43) Pub. Date: May 2, 2019

(54) COMPOUND FOR ENHANCING ACTIVITY OF ANTIBIOTIC COMPOSITIONS AND OVERCOMING DRUG RESISTANCE

(71) Applicant: SHOOLINI UNIVERSITY OF BIOTECHNOLOGY AND MANAGEMENT SCIENCES, Solan, Himachal Pradesh (IN)

(72) Inventors: Farooq UMAR, Solan, Himachal Pradesh (IN); Rana TANUJA, Solan, Himachal Pradesh (IN); Kaur NAVROOP, Solan, Himachal Pradesh (IN)

(73) Assignee: SHOOLINI UNIVERSITY OF BIOTECHNOLOGY AND MANAGEMENT SCIENCES, Solan, Himachal Pradesh (IN)

(21) Appl. No.: 15/571,651

(22) PCT Filed: May 3, 2016
(86) PCT No.: PCT/IN2016/000115

§ 371 Date: Nov. 3, 2017

(30) Foreign Application Priority Data
May 4, 2015 (IN) 1229/DEL/2015

(51) Int. Cl. A61K 31/235; A61P 31/04

(52) U.S. Cl. CPC A61K 31/235; A61K 31/496; A61P 31/04

(57) ABSTRACT

The present invention discloses a compound—‘ethyl gallate’ which can be used as an activity enhancer for enhancing the activity of antibiotic compositions and also overcoming drug resistance in bacteria. The compound was tested against gram negative, pathogenic bacteria viz. Pseudomonas aeruginosa and Klebsiella pneumoniae in combination with tetracycline, ciprofloxacin and chloramphenicol and found to be highly effective. The effect of the compound is due to its inhibitory action on the efflux pump of the bacteria, due to which even lower concentrations of the antibiotic compositions are highly effective in killing the bacteria. The compound thus offers the technical benefits of enhancing effects of antibiotic compositions at lower dosages and helping to overcome drug resistance in bacteria.

### FIELD OF INVENTION

The invention relates to the field of microbiology. The invention discloses a compound ‘ethyl gallate’ which is effective in enhancing the activity of antibiotic compositions and enabling their action against drug resistant bacteria i.e. overcoming drug resistance. The compound is a novel ‘Efflux Pump Inhibitor (EPI)’ which when co-administered with antibiotic compositions, enhances the activity of the antibiotic compositions and also helps to overcome bacterial resistance.

### BACKGROUND OF THE INVENTION

Antibiotics have been effective tools in the treatment of infectious diseases during the last half-century. From the development of antibiotic therapy to the late 1980s there was almost complete control over bacterial infections in developed countries. However, in response to the pressure of antibiotic usage, multiple resistance mechanisms have become widespread and are threatening the clinical utility of antibacterial therapy. The consequence of the increase in resistant strains is higher morbidity and mortality caused by bacterial infections.

Antibiotic Resistance Mechanisms in Bacteria

Bacteria have developed several different mechanisms to overcome the action of antibiotics. These mechanisms of resistance can be specific for a molecule or a family of antibiotics, or can be non-specific and be involved in resistance to unrelated antibiotics. Several mechanisms of resistance can exist in a single bacterial strain. These may act independently or they may act synergistically to overcome the action of an antibiotic or a combination of antibiotics.

Specific mechanisms include:
Degradation of the drug
Inactivation of the drug by enzymatic modification and
Alteration of the drug target

General mechanisms include:
Preventing access of the antibiotic to target by preventing or reducing transport of antibiotic into the cell
Increasing efflux of the drug from the cell to the outside medium

Both mechanisms can lower concentration of drug at the target site and allow bacterial survival in the presence of one or more antibiotics that would otherwise inhibit or kill the bacterial cells. Some bacteria utilize both mechanisms, combining a low permeability of the cell wall (including membranes) with an active efflux of antibiotics. In MDR (Multi drug resistant) bacteria, the over-expression of efflux pumps contributes to the reduced susceptibility by decreasing the intracellular concentration of antibiotics.

Efflux Pumps:

Efflux pumps are transport proteins involved in the extrusion of toxic compounds like antibiotics and present in both type of bacteria i.e., Gram-positive and Gram negative and even in some eukaryotic cells. Efflux is the mechanism in which bacteria transport compounds outside the cell wall which are potentially toxic, such as drugs or chemicals. Most of the efflux systems in bacteria are non-drug-specific proteins and can recognize and pump out a broad range of chemically and structurally unrelated compounds from bacteria in an energy-dependent manner. Because of their overwhelming presence in pathogenic bacteria, these active multi-drug efflux mechanisms are a major area of intense study.

In Bacteria there are Five Major Families of Efflux Transporters:

1. MF (major facilitator)

2. MATE (multidrug and toxic efflux)

3. RND (resistance-nodulation-division)

4. SMR (small multidrug resistance)

5. ABC (ATP binding cassette)

Overcoming drug resistance in bacteria

To fight with drug resistance three methods can be employed viz.
i. Development of new antibiotics.
ii. Use of plant extracts to enhance activity of existing drugs and overcome drug resistance
iii. Use of combination therapy in order to enhance activity of existing drugs and achieve bactericidal synergism.

Plants derived antimicrobials have been found to be activity enhancers. Though they may not have any antimicrobial properties alone, but when they are taken concurrently with standard drugs they enhance efficacy of existing drugs.

Efflux pump inhibitors (EPIs) are particularly the substances that give most promising approach in blocking the efflux pumps. They are the molecules which interfere with the process of removing toxic substances and antibiotics from the bacterial cell. Efflux pump inhibitors act as adjuvants to potentiate the activities of conventional antibiotics by inhibiting them either competitively or non-competitively.

Mode of Action
a) Alteration of configuration of pump due to direct binding to pump: The EPI (Efflux Pump Inhibitor) may bind directly to the pump in a competitive or non-competitive manner changing its shape and thus causing the blocking of the efflux pump and changing its activity.
b) Decrease in energy supplied to pump by binding to ATP: EPI may cause a depletion of energy, through the inhibition of the binding of ATP or the disturbance of the proton gradient across the membrane
c) Alteration of shape of substrate, making its efflux difficult: EPI may directly bind to the substrates forming a complex that facilitates the entry of the drug into the cell but prevents its efflux, allowing accumulation within the cell.

Efflux Pump Inhibitors:

The use of efflux pump inhibitors can facilitate the re-introduction of therapeutically ineffective (resistant) antibiotics back into clinical use and might even suppress the emergence of MDR strains. EPIs act synergistically and enhance the susceptibility of resistant antibiotics.

Synthetic EPIs Against Different Bacteria

Synthetic compounds remain to be the major approach in finding bacterial efflux pump inhibitors, because little is known about substrate-pump binding interaction. The use of synthetic EPI against different bacteria has been reviewed in literature:
1. Barrett J F (2001) has disclosed the use of L-phenylalanyl-L-arginyl-b naphthylamide (PAβN) as an efflux pump inhibitor to potentiate the activity of levofloxacin by 8 fold at against Pseudomonas aeruginosa (Microcide Pharmaceuticals, Curr. Opin. Investig. Drugs, 2(2), 2001, 212-215)
2. Mahamoud et al. (2006) have disclosed the use of quinoline as promising inhibitors of antibiotic efflux pump in multidrug resistant Enterobacter aerogenes isolates. Various quinoline derivatives significantly increased the intracellular concentration of chloramphenicol & thereby inhibit the transport of drug by AcrAB-TolC pump (Quinoline derivatives as promising inhibitors of antibiotic efflux pump in multidrug resistant Enterobacter aerogenes isolates, Cuff Drug Targets, 7(7), 2006, 843-847.

A lot of synthetic compounds have been worked out that work as Efflux Pump Inhibitors. However, only few like PAβN and CCCP (Carbonyl cyanide m-chlorophenylhydrazone) are found to be of some use and are the most common synthetic EPIs.

Plant Derived EPIs and their Limitations

Though a large number of synthetic and natural EPIs have been discovered, none have been approved for routine clinical use owing to doubtful clinical efficacy and high incidence of adverse effects.

Lorenzi et al. (2009) discloses that some of the plant extracts also show EPI like activity against Gram negative bacteria e.g: extracts of Helichrysum italicum, Thymus maroccanus, Thymus broussonetii and Callistemon citrinus enhanced the antimicrobial activity when combined with different antibiotics and they contain some EPI-like compounds that inhibit the efflux pumps of Pseudomonas aeruginosa (Geraniol restores antibiotic activities against multi drug resistant isolates, Gram-negative species. Antimicrob, Agents Chemother, 53(5), 2009, 2209-2211). However, the above EPIs are yet to be evaluated for clinical use.

The most popular natural occurring EPI is reserpine. Though it is active against many different efflux pumps viz. NorA, TetK, Bmrin Streptococcus pneumoniae, Staphylococcus aureus, Bacillus subtilis, a major limitation is that it needs to be used at high concentrations, which may cause toxicity at clinical levels.

Advantages of Present Invention:

The present invention overcomes limitations of prior art EPIs. It discloses a safe and effective EPI i.e. Ethyl gallate which is effective in enhancing the activity of antibiotic compositions at low dosages, thus negating concerns of toxicity associated with EPIs which have to be used at high concentrations. Secondly, the same acts as an activity enhancer for several antibiotics, enabling its wide use in several different antibiotic compositions. Thirdly, its use enhances activity of antibiotics not only against drug sensitive strains but also helps the same antibiotics to be effective against drug resistant strains. It thus offers the technical advantages of overcoming ‘drug resistance’ in a safe and effective manner, by making the existing antibiotic effective, than searching for new antibiotics.

### PRIOR ART

| S. no | Prior Art | Present Invention |
|---|---|---|
| 1. | U.S. Pat. No. 8,268,865 B2discloses synthetic, chemical | The present invention discloses a |
| | compounds - quaternary alkyl ammonium salts | natural, plant derived compound viz. a |
| | that inhibit bacterial efflux pump inhibitors | tannin Ethyl gallate and not any |
| | and are used in combination with an anti- | quaternary alkyl ammonium salts. The |
| | bacterial agent to treat or prevent bacterial | same is used as an EPI. |
| | infections. Does not disclose use of ethyl | |
| | gallate as an EPI. | |
| 2. | US 20130296228 A1 - discloses beta-lactam | Present invention does not disclose use |
| | compounds i.e. compounds a β-lactam ring in | of B-lactam compounds but discloses a |
| | their molecular structures. This includes | novel EPI inhibitor viz. Ethyl Gallate. |
| | penicillin derivatives (penams), | The same shows activity against |
| | cephalosporins(cephems), monobactams, and | specific efflux pump of gram negative |
| | carbapenems as efflux pump inhibitors and/or | bacteria and acts synergistically with |
| | porin modulators, which may be administered | three antibiotics (Ciprofloxacin, |
| | with antimicrobial agents for the treatment of | tetracycline and chloramphenicol). |
| | infections caused by various microorganisms, | |
| | in particular drug resistant microorganisms. | |
| 3. | US 20110117071 A1 - discloses use of | Use of ethyl gallate as an EPI inhibitor |
| | hydrogen peroxide as an EPI inhibitor when | is disclosed. |
| | combined with antimicrobial agents. | |
| 4. | Askoura et al. 2011 - discloses use of | Use of ethyl gallate as an EPI inhibitor |
| | Peptidomimetic compounds such as | is disclosed. No peptidomimetic |
| | phenylalanine arginyl b-naphthylamide | compounds are used. |
| | (PAbN) as efflux pump inhibitors (EPIs), No | |
| | mention of ethyl gallate at all. | |
| | (Ref: Efflux pump inhibitors (EPIs) as new | |
| | antimicrobial agents against Pseudomonas | |
| | aeruginosa Libyan J Med 2011, 6: 5870 - | |
| | DOI: 10.3402/ljm.v6i0.5870) | |

From the above it is clear that none of the prior art discloses the use of ethyl gallate as an efflux pump inhibitor, for enhancing the activity of antibiotic compositions against bacteria, including drug resistant bacteria.

In present invention, ethyl gallate has been tested against gram negative, pathogenic bacteria viz. Pseudomonas aeruginosa and Klebsiella pneumoniae.

Addition of this compound offered two technical benefits which are not disclosed in prior art viz.
i. Reduced the dose of antibiotics drastically owing to activity enhancing effect
ii. Made the antibiotics effective against even drug resistant bacteria.

### OBJECTS OF THE PRESENT INVENTION

The primary object of the present invention is to disclose use of ethyl gallate as an activity enhancer for antibiotic compositions.

Yet another object of the present invention is to disclose use of ethyl gallate as an Efflux Pump Inhibitor (EPI) which when co-administered with antibiotic compositions, helps to overcome drug resistance.

One more object of the present invention is to disclose a pharmaceutical composition comprising ethyl gallate and antibiotic, which has enhanced activity.

Still another object of the present invention is to disclose use of ethyl gallate or its derivatives as efflux pump inhibitors enabling wide use for enhancing activity of several antibiotic compositions in a safe and effective manner and also overcoming bacterial resistance.

### SUMMARY OF THE INVENTION

The present invention discloses a compound—‘ethyl gallate’ which can be used as an activity enhancer, for enhancing the activity of antibiotic compositions and also overcoming bacterial resistance to the antibiotics. The compound was tested against gram negative, pathogenic bacteria viz. Pseudomonas aeruginosa and Klebsiella pneumoniae in combination with tetracycline, ciprofloxacin and chloramphenicol and found to be highly effective. The effect of the compound is due to its inhibitory action on the efflux pump of the bacteria, because of which even lower concentrations of the antibiotic compositions are highly effective in killing the bacteria. The compound thus offers the technical benefits of enhancing effects of antibiotic compositions at lower dosages and overcoming drug resistance in bacteria.

### DESCRIPTION OF THE DRAWINGS

FIG. 1: Structure of ethyl gallate

### DETAILED DESCRIPTION OF THE INVENTION

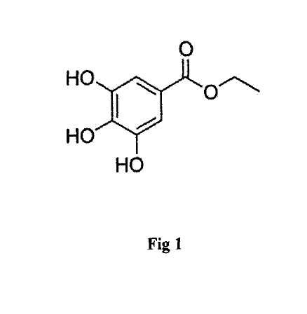

In the present invention, use of a plant derived compound (phytocompound) Ethyl gallate as an activity enhancer of antibiotic compositions, is disclosed. The compound is extracted from the leaves of the plant Terminalia chebula by alcoholic extraction. Its structure is given in FIG. 1.

Method of Extraction of Compound

2 kg dry powdered fruits were taken. Powdered dry fruits of T. chebula were extracted with methanol and dried till the moisture content was 7-8%. 1.6 liters of alcohol was added and refluxed three times at a temperature of 50° C. and vacuum 225 mm-Hg. The extract was steam distilled, followed by chemical fractionation.

Then column chromatography was performed on silica gel to extract the compound. 6 fractions for T. chebula plant were obtained by column chromatography. All fractions of T. chebula were subjected for their synergistic activity. The fraction showing synergistic activity was then characterized to know the physical properties of bioactive molecule and the molecular weight of bioactive molecule was determined by LCMS and the structure was elucidated by NMR. The compound extracted from T. chebula was colorless and soluble in methanol and water.

Its molecular weight was 198.17 and melting point was 150° C. with empirical formula C₉H₁₀O₅. Based on the molecular data obtained, the compound was identified as ethyl gallate and evaluated for bioactivity as below:

Bioactivity Testing

This was carried out to assess the potential of the compound to enhance antimicrobial activity and overcome antibacterial resistance. The 3 experimental groups used for bioactivity assessment of ethyl gallate are given in Table 1 below:

TABLE 1
Bioactivity assessment of Ethyl gallate
as activity enhancer of antibiotics

| | | Concentration at which the |
|---|---|---|
| Group | Description | compound was tested |
| A | Ethyl gallate alone | 1000 μg/ml |
| B | Antibiotics alone | Tetracycline = 30 μg/ml, |
| | | Ciprofloxacin = 5 μg/ml |
| | | Chloramphenicol = 30 μg/ml |
| C | Ethyl gallate + antibiotics | Ethyl gallate - 1000 μg/ml + |
| | | Tetracycline = 30 μg/ml or |
| | | Ciprofloxacin = 5 μg/ml or |
| | | Chloramphenicol = 30 μg/ml |

Strains tested
Klebsiella pneumonia (Drug sensitive and drug resistant)
Pseudomonas aeruginosa (Drug sensitive and drug resistant)

Drug Resistant Strains:

K. pneumonia isolate was collected from Gian Sagar medical college, Patiala and characterized in Immuno-parasitoloy Lab, Shoolini University, Solan for multidrug resistance. P. aeruginosa drug resistant strain was collected from Gian Sagar medical college, Patiala and characterized in Immunoparasitoloy Lab, Shoolini University, Solan.

Drug Sensitive (Control) Strains:

The resistant & sensitive control strains of K. pneumonia were procured from Dr. Enrique Llobet, CIBERES, Bunyola, Spain, Dr. Mazzariol of Verona University, Italy and one sensitive control strain were procured from IMTECH, Chandigarh (Table-2). Pseudomonas aeruginosa control cultures (Sensitive & Resistant) were procured from Dr. Thilo Kohler, University of Geneva, and Department of Microbiology and Molecular Medicine Geneve, Switzerland and one sensitive control strain were procured from IMTECH, Chandigarh (Table-3).

TABLE 2
Description of strains of Klebsiella pneumonia (resistant and sensitive)
used in the study

| | | |
|---|---|---|
| | Strain | |
| S. No. | name | Strain description |

RESISTANT STRAINS

| | | |
|---|---|---|
| 1. | 1740 | K. pneumonia AcrAB efflux pump repressor gene knockout strain, |
| | | was obtained from Dr. Enrique Llobet, CIBERES, Bunyola, Spain. |
| 2. | KLPN86 | K. pneumonia AcrAB efflux pump overexpressing strain, was |
| | | obtained from Dr. Mazzariol of Verona University, Italy. |
| 3. | KLPN105 | K. pneumonia AcrAB efflux pump overexpressing strain was |
| | | obtained from Dr. Mazzariol of Verona University, Italy. |
| 4. | KC4 | K. pneumoniae, clinical multidrug isolate was obtained from |
| | | GianSagar Medical College Patiala, Punjab, India. |
| 5. | KC18 | K. pneumoniae, clinical multidrug isolate was obtained from Gian |
| | | Sagar Medical College Patiala, Punjab, India. |

SENSITIVE STRAINS (CONTROL)

| | | |
|---|---|---|
| 1. | 1739 | K. pneumonia AcrAB efflux pump regulator gene knockout strain |
| | | was obtained from Dr. Enrique Llobet, CIBERES, Bunyola, Spain. |
| 2. | MTCC109 | K. pneumonia standard strain was obtained from IMTECH |
| | | Chandigarh. |
| 3. | 52145 | K. pneumonia AcrABwild type strain, was obtained from Dr. |
| | | Enrique Llobet, CIBERES, Bunyola, Spain. |

TABLE 3
Description of strains of Pseudomonas aeruginosa used in the study

| | | |
|---|---|---|
| | Strain | |
| S. No. | name | Strain description |

RESISTANT STRAINS

| | | |
|---|---|---|
| 1. | R2 | PAO1 derivative, overexpressing strain of |
| | | mexAB-oprM, resistant strain-Positive control |
| 2. | R3 | Wild type strain of mexAB-oprM pump, resistant |
| | | strain-Positive control |
| 3. | R4 | PAO1 nalB derivative, 2 bp deletion in mexR, |
| | | overexpresses mexAB-oprM, resistant |
| | | strain-Positive control |
| 4. | Ps-3 | Pseudomonas aeruginosa MDR Clinical Isolate (Ps3)- |
| | | Resistant strain |
| 5. | Ps-T10 | Pseudomonas aeruginosa MDR Clinical Isolate |
| | | (PsT10)-Resistant strain |
| 6. | Ps-11 | Pseudomonas aeruginosa MDR Clinical Isolate |
| | | (Ps11)-Resistant strain |

SENSITIVE STRAINS (CONTROL)

| | | |
|---|---|---|
| 1. | MTCC- | Pseudomonas aeruginosa (MTCC-471) (Standard) |
| | 471 | Sensitive strain |
| 2. | R1 | Derivative of TETR mutated in oprM by insertion of |
| | | Hg cassette, hyper susceptible strain)-Negative control |

Results obtained with different strains indicated significant potential of ethyl gallate as a ‘activity enhancer’ of antibiotic compositions, against not only drug sensitive strains but also against drug resistant strains.

Tables below illustrate the results obtained.

TABLE 4
Use of Ethyl Gallate as activity enhancer of
antibiotics using K. pneumonia

| | | K. pneumonia | |
|---|---|---|---|
| | | Strain 52145 | |
| | | (Drug sensitive | |
| | | or control) | K. pneumonia |
| | | Minimum | 1740 |
| | Concentration of | Inhibitory | knockout |
| | antibiotics and enhancers | Concentration | (Drug resistant) |
| Group | used | (MIC)μg/ml | MICμg/ml |
| A | Ethyl gallate alone | 2.000 | 2.000 |
| | 1000 μg/ml | | |
| B | Antibiotics alone | | |
| | Ciprofloxacin = 5 μg/ml | 0.060 | 0.125 |
| | Tetracycline = 30 μg/ml | 2.000 | 2.000 |
| | Chloramphenicol = 30 μg/ml | 2.000 | 2.000 |
| C | Ethyl gallate + antibiotics | | |
| | Ethyl gallate 1000 + | | |
| | antibiotics | | |
| | Ciprofloxacin = 5 μg/ml | 0.003 | 0.003 |
| | Tetracycline = 30 μg/ml | 0.500 | 0.500 |
| | Chloramphenicol = 30 μg/ml | 1.000 | 0.500 |

Interpretation of the Results

Ethyl gallate itself is having good antimicrobial activity, comparable to that of tetracycline and chloramphenicol (MIC=2 micrograms/ml). However, when used in combination with these antibiotics, it enhances the activity of the antibiotics drastically (2 fold to 40 fold).

In Case of Drug Sensitive Strain of K. pneumonia:

Ethyl gallate drastically reduced the concentration of antibiotic required in all three cases viz. ciprofloxacin, tetracycline and chloramphenicol. The decrease ranged from 2 fold to 40 fold as given in Table 5 below.

TABLE 5
Activity enhancement of antibiotics on addition of ethyl gallate
(decrease in antibiotic concentration)

| | | | |
|---|---|---|---|
| MIC | MIC | MIC | Fold decrease in |
| Antibiotic | Ethyl | Antibiotic + | antibiotic |
| Alone | Gallate | ethyl gallate | concentration |

CIPROFLOXACIN (5 μg/ml)

| | | | | |
|---|---|---|---|---|
| Sensitive Strain* | 0.060 | 2.000 | 0.003 | 20 |
| Resistant Strain** | 0.125 | 2.000 | 0.003 | 40 |

TETRACYCLINE (30 μg/ml)

| | | | | |
|---|---|---|---|---|
| Sensitive Strain* | 2.000 | 2.000 | 0.500 | 4 |
| Resistant Strain** | 2.000 | 2.000 | 0.500 | 4 |

CHLORAMPHENICOL (30 μg/ml)

| | | | | |
|---|---|---|---|---|
| Sensitive Strain* | 2.000 | 2.000 | 1.000 | 2 |
| Resistant Strain** | 2.000 | 2.000 | 0.500 | 4 |

*K. pneumonia Strain 52145(Drug sensitive)
**K. pneumonia 1740 knockout (Drug resistant)

TABLE 6
Use of Ethyl Gallate as activity enhancer of antibiotics using P. aeruginosa

| | | P. aeruginosa Strain | |
|---|---|---|---|
| | | MTCC-471 | P. aeruginosa |
| | | (Drug sensitive or control) | PS11 |
| | Concentration of antibiotics | Minimum Inhibitory | (Drug resistant) |
| Group | and enhancers used | Concentration (MIC) μg/ml | MIC μg/ml |
| A | Ethyl gallate alone | 0.06 | 2.00 |
| | 1000 μg/ml | | |
| B | Antibiotics alone | | |
| | Ciprofloxacin = 5 μg/ml | 0.125 | 2.00 |
| | Tetracycline = 30 μg/ml | 0.125 | 2.00 |
| | Chloramphenicol = 30 μg/ml | 1.000 | 2.00 |
| C | Ethyl gallate + antibiotics | | |
| | Ethyl gallate 1000 + antibiotics | | |
| | Ciprofloxacin = 5 μg/ml | 0.060 | 1.00 |
| | Tetracycline = 30 μg/ml | 0.060 | 0.50 |
| | Chloramphenicol = 30 μg/ml | 0.050 | 1.00 |

Ethyl gallate drastically reduced the concentration of antibiotic compositions required in all three cases viz. ciprofloxacin, tetracycline and chloramphenicol. The decrease ranged from 2 fold to 40 fold as given in Table 7 below.

TABLE 7
Activity enhancement of antibiotics on addition of ethyl gallate
(decrease in antibiotic concentration)

| | | | |
|---|---|---|---|
| MIC | MIC | MIC | Fold decrease in |
| Antibiotic | Ethyl | Antibiotic + | antibiotic |
| Alone | gallate | ethyl gallate | concentration |

CIPROFLOXACIN (5 μg/ml)

| | | | | |
|---|---|---|---|---|
| Sensitive Strain* | 0.125 | 0.060 | 0.060 | 2 |
| Resistant Strain** | 2.000 | 2.000 | 1.000 | 2 |

TETRACYCLINE (30 μg/ml)

| | | | | |
|---|---|---|---|---|
| Sensitive Strain* | 0.125 | 0.060 | 0.060 | 2 |
| Resistant Strain** | 2.000 | 2.000 | 0.500 | 4 |

CHLORAMPHENICOL (30 μg/ml)

| | | | | |
|---|---|---|---|---|
| Sensitive Strain* | 1.000 | 0.060 | 0.050 | 20 |
| Resistant Strain** | 2.000 | 2.000 | 1.000 | 2 |

*P. aeruginosa Strain MTCC-471 (Drug sensitive)
**P. aeruginosa PS11 (Drug resistant)

Hence it can be concluded that Ethyl gallate acts as an activity enhancer and addition of this compound reduced the dose of antibiotic compositions drastically owing to activity enhancing effect and made the antibiotics effective against even drug resistant bacteria.

### NOVELTY, INVENTIVE STEP AND INDUSTRIAL APPLICATION OF THE INVENTION

Novelty

The novelty of the present invention lies in disclosing an activity enhancer for antibiotics, which when added to antibiotic compositions greatly enhances their activity and also helps to overcome antibiotic resistance. The compound is ethyl gallate extracted from the fruit of the plant Terminalia chebula and can be easily added to existing or known antibiotic compositions to enhance their activity. Use of ethyl gallate as an activity enhancer has not been disclosed in the prior art.

Inventive Step

The technical advancement of knowledge lies in disclosing a compound, Ethyl gallate which can not only be used to enhance the activity of existing antibiotic compositions but also overcoming drug resistance, in a safe and effective manner. Method of extracting the compound and assessment of its antimicrobial activity is also disclosed. The compound has been shown to be effective in enhancing activity and also overcoming bacterial resistance when added to three antibiotics viz. ciprofloxacin, tetracycline and chloramphenicol.

### INDUSTRIAL APPLICATION

Present invention has widespread application in pharmaceuticals and clinical fields owing to therapeutic benefits of enhancing antibiotic effectiveness and also overcoming bacterial resistance. Industrial application of this compound is facilitated because there are no concerns of toxicity or undesirable side-effects because the same is already an approved food additive with E number E313. It is the ethyl ester of gallic acid and is produced from gallic acid and ethanol. It is added to food as an antioxidant. Additionally, it is a compound which is found naturally in a variety of edible plant sources including walnuts, Terminalia myriocarpa, or chebulicmyrobolan (Terminalia chebula).

1. A compound for enhancing the activity of antibiotic compositions and enabling their action against drug resistant bacteria also wherein the compound is ethyl gallate.

2. The compound as claimed in claim 1 wherein addition of the same to an antibiotic composition in appropriate ratios enhances the activity of the composition by 2 to 40 fold in terms of minimum inhibitory concentration of the antibiotic composition.

3. The compound as claimed in claim 1 which when added to the antibiotic ciprofloxacin in appropriate ratio, enhances its antimicrobial activity against drug sensitive as well as drug resistant bacteria.

4. The compound as claimed in claim 1 which when added to the antibiotic tetracycline enhances its antimicrobial activity against drug sensitive as well as drug resistant bacteria.

5. The compound as claimed in claim 1 which when added to the antibiotic chloramphenicol enhances its antimicrobial activity against drug sensitive as well as drug resistant bacteria.

6. The compound as claimed in claim 1 wherein the same is effective in enhancing the activity of antibiotics against drug sensitive and drug resistant strains of Klebsiella pneumonia.

7. The compound as claimed in claim 1 wherein the same is effective in enhancing the activity of antibiotics against drug sensitive and drug resistant strains of Pseudomonas aeruginosa.
